(19) United States
(12) Patent Application Publication
(10) Pub. No.: US 2022/0133432 A1
(43) Pub. Date: May 5, 2022

(54) VACCINATION VERIFICATION BRACELET

(71) Applicant: Marian Elizabeth Guirguis, Indianapolis, IN (US)

(72) Inventor: Marian Elizabeth Guirguis, Indianapolis, IN (US)

(21) Appl. No.: 17/500,492

(22) Filed: Oct. 13, 2021

Related U.S. Application Data
(60) Provisional application No. 63/091,416, filed on Oct. 14, 2020.

(51) Int. Cl. A61B 90/96; G09F 3/00; G16H 10/65

(52) U.S. Cl. CPC A61B 90/96; G16H 10/65; G09F 3/0297; G09F 3/005

(57) ABSTRACT

Vaccine status verification devices and systems for third parties to determine and verify the vaccination status of a user. In one embodiment, information relating to the vaccination status, vaccination date(s), and optionally other information related to a user is stored on and displayable from a bracelet worn by the user.

### CROSS REFERENCE TO RELATED APPLICATIONS

This application is related to and claims priority to U.S. Provisional Patent No. 63/091,416 filed on Oct. 14, 2020, which is incorporated by reference herein.

### BACKGROUND

The COVID-19 pandemic has underscored the importance of preventative medicine. As the pandemic has progressed and vaccines have been developed and distributed, it has become increasingly important to be able to show proof of vaccination in order to gain maskless access to certain establishments and venues. However, there is no universal way to prove vaccination. Different vaccination entities provide different methods of proof of vaccination. Most provide cards, but the cards are not standardized in size, format and/or content and tend to not be especially wear-resistant. Further, it is difficult or impossible to get a replacement card for one that is lost or simply worn out. Thus, there remains a very pressing need for a standardized and wear-resistant means for providing proof of vaccination. The present novel technology addresses this need.

### BRIEF DESCRIPTION OF DRAWINGS

FIG. 1 is a front plan view of a vaccination verification bracelet according to one example of the disclosed technology.

FIG. 2 is a side view of the bracelet shown in FIG. 1.

FIG. 3 is a rear plan view of the bracelet shown in FIG. 1.

FIG. 4 is a front plan view of a vaccination bracelet according to another example of the disclosed technology.

FIG. 5 is a schematic view of a vaccination verification system according to one embodiment.

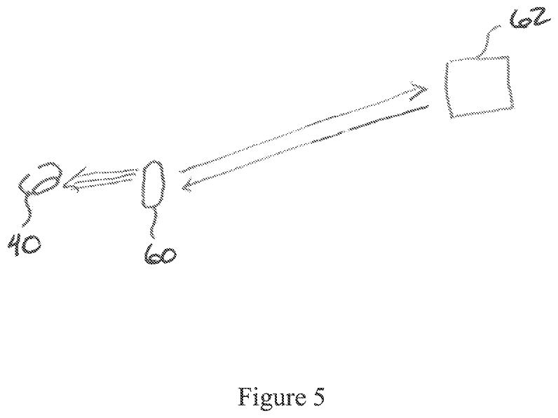

### DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

The claimed technology provides a new method and device for quickly and reliably ascertaining whether the vaccination record of a user. In some embodiments the claimed technology is proactive in information sharing so that the user automatically shares vaccination confirmation with a venue or establishment user's device is queried ad responds with vaccination confirmation and/or more detailed vaccination and like medical history. In other embodiments, the claimed technology may have global application. In still other embodiments, the technology disclosed herein may be adapted for use with respect to other types of infectious diseases and/or vaccines such as Middle East Respiratory Syndrome (MERS), Severe Acute Respiratory Syndrome (SARS), Influenza, Measles, Mumps, and Rubella (MMR), and the like. In further embodiments, the disclosed technology may be adapted for use with other types of jewelry or wearable items such as necklaces, pendants, broches, and the like.

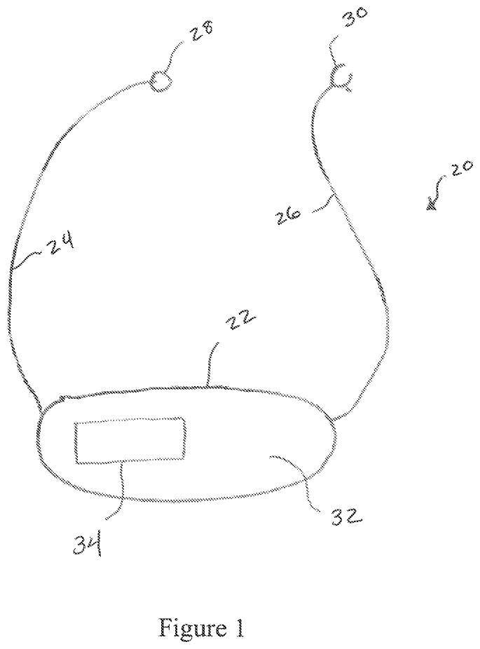

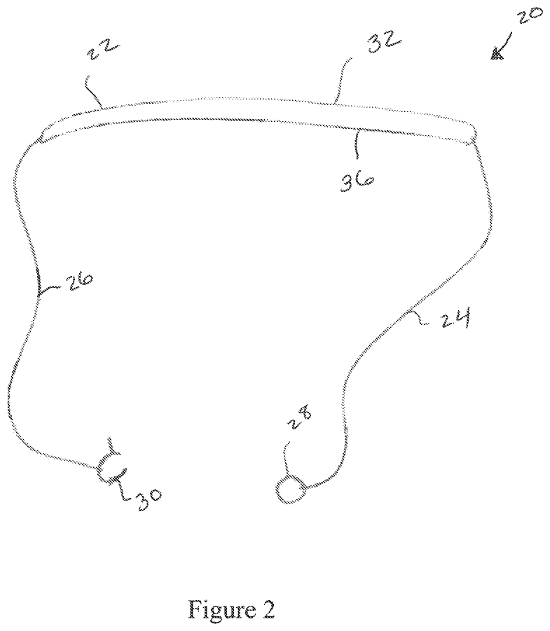

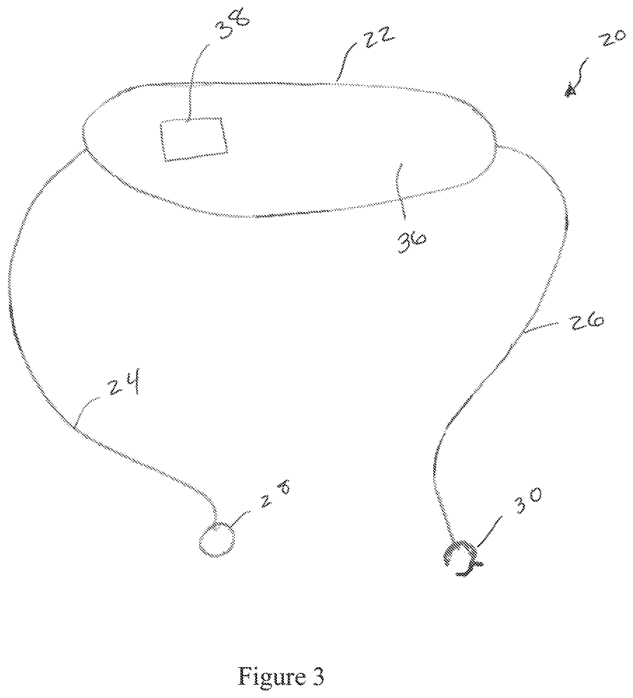

FIGS. 1-3 show a vaccination verification bracelet 20 according to one example of the disclosed technology. In this particular example, the bracelet 20 includes a plate portion 22, a first band or strap 24 coupled to a first clasp portion 28, and a second band or strap 26 coupled to a second clasp portion 30. The exact length and construction of the first strap 24 and second strap 26 may vary as desired. The straps may be made from metal (stainless steel, gold, silver, alloys thereof, and the like), natural materials (leather, hemp, jute, cotton, and the like), synthetic materials (synthetic rubber, vinyl, plastics, and the like), composites, and combinations thereof. The exact style of the straps may include individual links, braided material, woven strands, or other suitable construction techniques. In still other examples, the straps may be a single continuous band which is made from a flexible, stretchable material to allow it to be slipped over a user's wrist. The clasp portions 28, 30 may be a variety of different suitable devices or coupling and uncoupling bracelets such as spring rings (as shown in FIGS. 1-3), barrels, bayonet style, open box, FIG. 8 safety clasp, S-hook, magnetic, pearl style, and the like. The exact length of the strap portions 24, 26 may vary as desired to fit users having different hand and wrist sizes. Optionally, the strap portions 245, 26 may be removeable and replaceable if they become worn, broken, or need to be increased in size.

The plate portion 22 includes a front face 22 and a rear face 36. The front face 32 includes an information display area 34 and the rear face 36 includes an information display area 38 in which information related to a user's vaccination status may be displayed. The exact size and shape of the display areas 34, 38 may vary, but typically will be large enough to display the desired information so it can be easily read. Information may be recorded in one of both of the information display areas using a variety of suitable recording means such as engraving, chemical etching, laser etching, painting, material additive techniques such as jetting, direct deposition, and the like. Different colors may be used for displaying information so as to make some information easy to read (e.g., using highly contrasting colors) and other information more difficult to read (e.g., using low contrasting colors). Optionally, one or both display areas may be larger than required to contain and display the information initially recorded therein so as to allow for the addition of further information at a later time. For example, an information display area may be size so as to include enough area so that vaccination booster information may be added later when such a booster is received by a user. The exact information shown in the display areas 34, 38 may include a user's name, the fact that a user has been fully or partially vaccinated, the name of the Covid-19 vaccine they have received, the date(s) on which the vaccine was administered, where the vaccine was administered, and/or the name and contact information (phone, text, email, and the like) for the entity which administered the vaccination. In other examples, the information display areas may include images such as an etched image of a vaccination card. Optionally, the information display areas 34, 38 may display the same or different information. For example, the information display area 34 on the front fact 32 of the bracelet 20 might include a generic statement that the wearer has been fully vaccinated while more personal information (the user's name, the date(s) the vaccine was administered, etc.) might only be displayed on the information display area 38 on the rear face of the bracelet 36 so as to not be visible by casual observers but would still be easily accessible by a user to produce and display when asked for proof of vaccination.

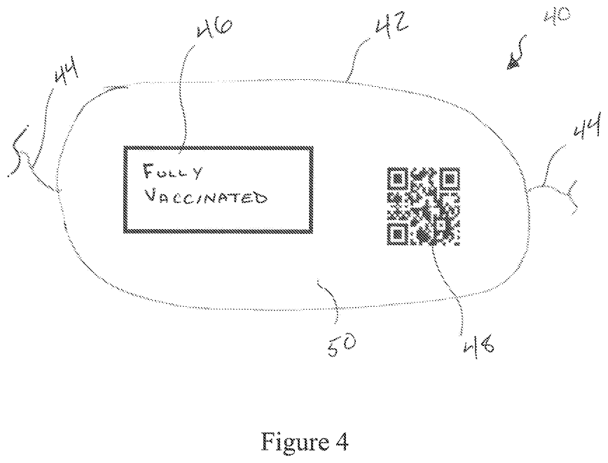

A bracelet 40 according to another embodiment is shown in FIG. 4. In this particular example, the bracelet 40 includes a plate portion 42 connected to a strap portion 44. The exact size and nature of the strap portion 44 may vary as desired and has been omitted in this view for clarity. The plate portion 42 has a front face 50 which has a first information display area 46 and a second information display area 48. In this particular example, the first information display area 46 is suitable for displaying text and the second information display area 48 is suitable for displaying scannable information. The exact nature of the scannable information varies, but may include a quick response code (QR code) such as that shown in FIG. 4 or other suitable linear or matrix data displays (bar codes). In this example, the first information display area 46 might display generic information concerning the wearer such as that they are fully vaccinated while the second information display area 48 might contain a code which when scanned using a suitable reading device 60 (phone, table, computer, etc.) would allow a user to access remotely stored data 62 which is stored on a suitable remote device(s) such as a particular computer, server, or on a cloud storage system or service and which includes more detailed information about the wearer. In one example, the remotely stored data might include an image of the wearer's vaccination card, an image of the wearer's photo identification (to prevent people from sharing bracelets), contact information for the wearer, contact information for the entity which vaccinated the wearer. In this particular example, the use of a digital information display area allows for a single bracelet to be used long term even as more medical and vaccination information is generated by a wearer such as when they receive a vaccination booster for Covid-19 or when they receive vaccination for other infectious diseases such as tuberculosis or influenza. Optionally, the digital information display area may be associated with a wearer's testing information and results so as to display the date and results of a wearer's latest Covid-19 test. In still other examples the digital information display area may be associated with other testing information of a wearer such as HIV status, tuberculosis status, hepatitis status, and the like.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. A device for storing and displaying information relating to the vaccination status of a user, comprising:
a bracelet having a plate portion and a strap portion; and
the plate portion having a front face with a front display area and a rear face with a rear display area;
wherein information related to the vaccination status of a user is displayed in at least one of the front display area and rear display area.

2. The device of claim 1, wherein the front face further includes a second front display area.

3. The device of claim 2, wherein the second front display area includes scannable information displayed as a matrix data display.

4. The device of claim 1 wherein the information related to the vaccination status of the user includes one or more of the user's name, type of vaccine received, date of vaccination, date of a Covid-19 test, and results of a Covid-19 test.

5. The device of claim 1, wherein the front display area indicates the user has been vaccinated and the rear display area includes one or more of the user's name, type of vaccine received by the user, date of vaccination of the user, date of a Covid-19 test on the user, and results of a Covid-19 test of the user.

6. A system for verifying the vaccination status of a user, comprising:
providing a vaccination status bracelet to a user having a plate portion with a front display area and a rear display area;
recording information relating to the vaccination status of the user in one or both of the front display area and rear display area;
wearing the vaccination status bracelet on the user's wrist; and
displaying one or both of the front and rear display area to confirm the vaccination status of the user.

7. The system of claim 6, wherein the front display area and the rear display area have the same information recorded therein.

8. The system of claim 6, wherein the front display area and the rear display area have different information recorded therein.

9. The system of claim 8, wherein the information recorded in the front display area is that the user is vaccinated and the information recorded in the rear display area is one or more of the user's name, type of vaccine received by the user, date of vaccination of the user, date of a Covid-19 test on the user, and results of a Covid-19 test of the user.

10. The system of claim 6, wherein the front plate portion further includes a second display area.

11. The system of claim 10, wherein the second front display area includes scannable information displayed as a matrix data display.
